(19) United States
(12) Patent Application Publication
(10) Pub. No.: US 2024/0423449 A1
(43) Pub. Date: Dec. 26, 2024

(54) ENDOSCOPE COMPRISING A FLUID PATHWAY SYSTEM

(71) Applicant: AMBU A/S, Ballerup (DK)

(72) Inventor: Alistair David MORTON, Copenhagen (DK)

(73) Assignee: AMBU A/S, Ballerup (DK)

(21) Appl. No.: 18/742,925

(22) Filed: Jun. 13, 2024

(30) Foreign Application Priority Data
Jun. 23, 2023 (EP) 23181218.1

(51) Int. Cl. A61B 1/00

(52) U.S. Cl. CPC A61B 1/00066

(57) ABSTRACT

An endoscope and a system including the endoscope and a monitor. The endoscope includes a handle or interface; an insertion cord extending distally from the handle or interface; and a fluid pathway system having a first flow-through part having a fluid conduit formed therein and including a receiver portion; a second flow-through part having a fluid conduit formed therein and including a tubular connector portion; a retainer having an insertion end and a reception end, the insertion end of the retainer inserted in the receiver portion of the first flow-through part, the tubular connector portion of the second flow-through part inserted in the reception end of the retainer, and the retainer being sized and configured to fluidly sealingly secure the second flow-through part to the first flow-through part.

### CROSS-REFERENCE TO RELATED APPLICATIONS

This application claims priority from and the benefit of European Patent Application No. 23181218.1, filed Jun. 23, 2023; the disclosure of said application is incorporated by reference herein in its entirety.

### TECHNICAL FIELD

The present invention relates to endoscopes, and in particular endoscopes comprising a fluid pathway system.

### BACKGROUND

Endoscopes are known and used for visual navigation into, and examination and diagnosis of, hollow organs and body cavities, as well as, optionally, to assist in surgery, e.g. for targeted tissue sampling. Endoscopes include procedure-specialized endoscopes, such as gastroscopes and bronchoscopes. Endoscopes may comprise a handle at the proximal end to be gripped by an operator and a flexible, elongated insertion cord extending distally from the handle. The insertion cord may include an insertion tube terminated in a distal tip part at the end of a highly bendable, e.g. articulated, bending section controllable by the operator. The tip part may comprise an observation optical system extending distally from the bending section. Some endoscopes include a fluid pathway for transporting media into or out of the body, e.g. delivery of a medical fluid or aspiration to remove fluids from a target position inside the body. The fluid pathway generally runs inside the endoscope from a position near the proximal end of the endoscope, i.e. outside the body, to a position near the distal end of the endoscope, i.e. inside the body.

Reusable endoscopes need to be thoroughly cleaned between each patient, and the fluid pathways are notoriously difficult to clean and hence the cleaning process is time-consuming. Over time biofilm may form in the fluid pathways, which may make the cleaning process virtually impossible and constitute a potential health hazard.

Single-use endoscopes, on the other hand, do not need to be sterilized after use. For single-use endoscopes, it is important that the entire device is manufactured in a cost-efficient way. Single-use endoscopes in the market are very capable and are widely used, but even such endoscopes can be further improved.

Further, the endoscope should live up to high standards on the general performance of the endoscope, and it is desirable to improve versatility of endoscopes as well as reducing cost, particularly in single-use endoscopes, to increase their value.

### BRIEF DESCRIPTION OF THE DISCLOSURE

The objective of the present disclosure is to provide an endoscope with features that eliminate or at least reduce the disadvantages of the prior art and suitably deal with the problems mentioned above. In particular, an endoscope shall be provided, which is designed for single-use, and which has a fluid pathway system made of commercially available and low-cost materials. Preferably, the single-use endoscope should be made in such a way that it can be disassembled in a cost-effective manner and its component parts recycled to reduce the impact on climate and the environment.

A first aspect of this disclosure relates to an endoscope comprising a fluid pathway system comprising a first flow-through part, a second flow-through part, and a retainer having an insertion end and a reception end, the insertion end inserted in the first flow-through part, the reception end receiving the second flow-through part, and the retainer being sized and configured to fluidly sealingly secure the second flow-through part to the first flow-through part.

The endoscope according to the first aspect is beneficial in that it enables high and reliable flow performance by superior sealing and low pressure loss. As an example, the fluid pathway system may be used to provide suction at the endoscope distal tip by connecting to a vacuum source. Medical vacuum at hospitals is often provided at a pressure of approximately 50 kPa below atmospheric pressure. For best performance of the endoscope pressure loss in the fluid pathway system should be kept low. High suction capability of the endoscope may be used to have a clear sight by the observation optical system by removing blood or mucus.

The endoscope according to the first aspect has the advantage that the fluid pathway system is fast and easy to assemble. Further, the assembly does not require any special skills or education, as the assembly only requires that parts are pushed into engagement. The construction is also tolerant in that if parts are connected wrongly, they can be disassembled and reconnected, which lowers the scrap rate and hence lowers cost and environmental impact. An extra benefit is that the endoscope can be disassembled after use, which makes recycling of materials much more efficient and lowers the environmental impact. Further, the parts are not subject to high forces, which could over time otherwise lead to aging and increase a potential risk of disconnection or leaks.

In an embodiment according to the first aspect, the endoscope comprises a handle or interface, and an insertion cord extending distally from the handle or interface.

In a variation of the first embodiment, the first flow-through part has a fluid conduit formed therein and comprises a generally cylindrical receiver portion with a receiver inner diameter, the second flow-through part has a fluid conduit formed therein and comprises a tubular connector portion with a connector outer diameter, the retainer has a generally cylindrical outer surface and an inner space having a generally cylindrical inner surface, wherein the retainer has an inner retainer diameter of the inner space and an outer retainer diameter of the outer surface, the retainer inner diameter fitting the connector portion outer diameter and the retainer outer diameter fitting the receiver portion inner diameter, so that the retainer is configured to seal and secure the second flow-through part to the first flow-through part.

A flow-through part is any part provided with a fluid channel, such as a tube, a valve, a pipe connection, a pipe stub or a nozzle. In this context a “first flow-through part” is a part configured to receive a retainer and the “second flow-through part” is a part configured to be inserted into the retainer, i.e. the first flow-through part comprises a female connector, whereas the second flow-through part comprises a male connector. In some cases, the same part can constitute a “first flow-through part” as well as a “second flow-through part”, if the part is configured to provide connection to two other flow-through parts by having both a female connector and a male connector.

In one example of the present embodiment or the present variation, the retainer comprises smooth inner and outer surfaces. In another example of the present variation, however, the retainer comprises annular sealing protrusions on the inner surface and/or the outer surface. Annular sealing protrusions may provide for low friction insertion of the retainer and low friction uptake of parts in the retainer interior compared to a retainer having a smooth surface. Further annular sealing protrusions may add to the fluid tightness of the fluid pathway system. The retainer may have, for example, four annular protrusions, but it some cases it may suffice to have only one, two or three annular protrusions, whereas in other cases more than four annular protrusions may be provided, such as five to seven annular protrusions.

In one example of the present embodiment or the present variation, the first flow-through part is a Y-connector, and the second flow-through part is a suction tube.

In one example of the present embodiment or the present variation, the first flow-through part is a suction valve, and the second flow-through part is a suction tube and/or a vacuum hose fitting.

The endoscope may comprise an adhesive in the fluid pathway system, e.g. if considered expedient to avoid dislocation of the parts making up the fluid pathway system. It is preferred, however, that the fluid pathway system is essentially adhesive-free. That the fluid pathway system is essentially adhesive-free eliminates the risk of errors in production, such as adhesive flowing to locations, where it could negatively impact fluid flow. An adhesive-free system also has the benefit that it greatly increases the possible choice of materials, such as the use of silicone materials, which are inherently difficult to adhere to, and of course it also facilitates recycling by easy disassembly of parts.

The endoscope may incorporate a retainer made of any suitable material, but according to an embodiment the retainer is made of a material having a hardness in the interval of 10-100 Shore A, preferably 50-70 Shore A, which is considered to provide a suitable compromise between ease of assembly, sealing capability and grip. A very low hardness can make it difficult to produce and handle the retainer. The hardness is measured according to ISO 868:2003.

According to an embodiment, the retainer comprises a flange at the receiver end thereof. The flange may aid during insertion of the retainer and may further indicate full and correct insertion of the retainer as the flange may abut the end of the receiver portion of the first flow-through part.

According to an embodiment, the retainer comprises a lead-in zone to the inner space at the receiver end. The lead-in zone may facilitate insertion of the second flow-through part in the retainer, e.g. by providing a funnel-shaped entry to the retainer inner space.

According to an embodiment, the retainer comprises a constriction at the insertion end thereof. The constriction may provide a seal between the first and second flow-through parts.

In one example of the present variation, the inner interface between first flow-through part, second flow-through part and retainer constriction is configured to provide a consistent inner diameter.

To provide high sealing efficiency and grip, without assembly being too laborious, it is preferred that the retainer has a ratio of length to outer diameter in the range of 6:5 to 7:5. This interval is considered to provide good results. A ratio above 7:5 is possible and may improve sealing efficiency and grip of the retainer, but may make it more difficult to insert the retainer, e.g. because of longer distance to cover during insertion and potentially a risk of buckling of the retainer during insertion. A ratio below 6:5 is also possible, and may make insertion of the retainer easier, and potential disassembly after use easier, whereas potential lower sealing efficiency and grip of the retainer can be counteracted e.g. by adding further annular sealing protrusions, providing a tighter fit or using softer materials.

The retainer may be assembled of a plurality of parts, e.g. a separate shell part of a rigid material and separate flexible seal rings mounted thereon, however it is preferred that the retainer is molded in one piece from an elastic material, such as Liquid Silicone Rubber (LSR), preferably medical grade silicone. This is considered to be advantageous for cost of production and assembly, and medical grade silicone is considered safe to use in endoscopes. An example of a suitable material is LSR of the Elastosil® LR 3003 series. Alternatively, all thermoplastic elastomers may be used, which may have some advantages in terms of sustainability, as such materials can be easily recycled, and the retainer may with this material be produced by injection molded.

A second aspect of the present disclosure relates to a visualization system comprising an endoscope as described above, and a monitor connectable to the endoscope.

### BRIEF DESCRIPTION OF THE DRAWINGS

The disclosure will now be made in greater detail based on non-limiting exemplary embodiments and with reference to the schematic drawings on which:

FIG. 1 shows a side view of an endoscope connectable to a monitor to form a visualization system;

FIG. 2 shows an endoscope according to the disclosure and the interior of the handle thereof;

FIG. 3 shows a fluid pathway system according to the disclosure;

FIG. 4 is a perspective view of a retainer according to the disclosure;

FIG. 5 is a longitudinal section of the retainer of FIG. 4;

FIG. 6 is a longitudinal section of a detail of an assembled fluid pathway system corresponding to FIG. 3 and illustrating the retainer of FIG. 4 as fitted in the fluid pathway;

FIG. 7 is a sectional side view of a variation of the assembled fluid pathway system of FIG. 6;

FIG. 8 is an exploded sectional side view of the fluid pathway system of FIG. 6;

FIG. 9 is an exploded sectional side view of another variation of the fluid pathway system of FIG. 6; and

FIG. 10 is a sectional side view of another variation of a retainer.

The figures are schematic in nature and serve only to understand the disclosure. Identical elements are marked with the same reference signs.

### DETAILED DESCRIPTION

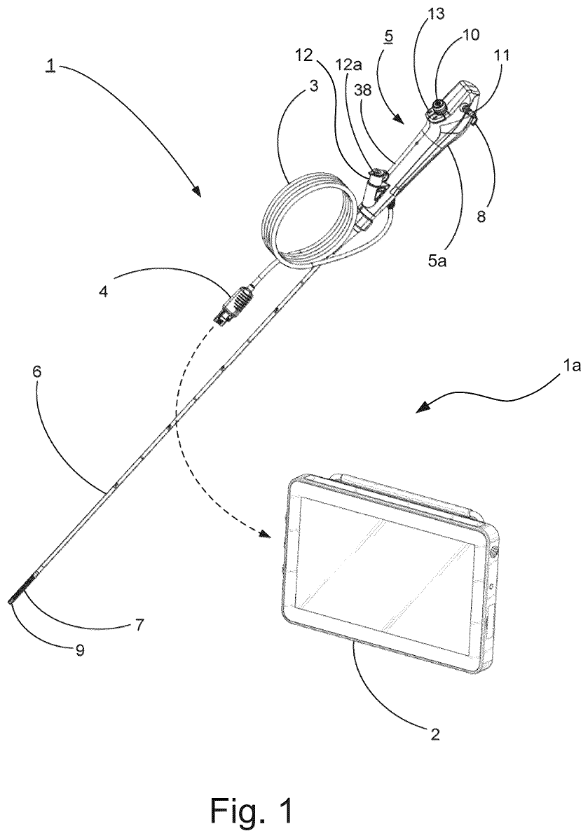

FIG. 1 illustrates a system 1a comprising an endoscope 1 and a monitor, or display unit, 2. The endoscope 1 comprises an umbilical cord 3 and an electrical connector 4 for connecting an image sensor of the endoscope 1 to the display unit 2. The endoscope 1 is preferably single-use i.e. intended to be thrown away after use on one single patient, whereas the display unit may 2 be used multiple times with different single-use endoscopes. The endoscope 1 further comprises a handle 5 at a proximal end of the endoscope and an insertion cord 6 extending distally of the handle 5. The insertion cord 6 comprises a bending section 7 and a tip part 9. The bending section 7 may be controlled by a steering lever, or controller, 8 to deflect the distal end of the insertion cord 6. At the distal end of the bending section 7 is the tip part 9. The tip part 9 includes an optical system that, generally, comprises a camera and a light source. The light source may comprise light emitting diodes (LEDs) or, in an alternative arrangement, optical fibers may be provided for transporting light from a light source at the handle or outside the endoscope through the endoscope to the distal end thereof for illuminating an area of interest in front or a side of the tip part 9.

The insertion cord 6 may comprise one or more channels, e.g. a working channel for advancing tools through the endoscope and out through an opening at the distal end of the endoscope, and a fluid pathway, such as a suction channel. The suction channel may be used for transporting fluids away from a position at the distal end of the endoscope, through an opening at the distal end of the endoscope. The same channel may be used as working channel and suction channel. The handle 5 often also comprises a suction valve button 10 comprising an external valve cap forming a push-button for activating suction through the suction channel to an external vacuum source (not shown) via a vacuum hose connector 11. The handle 5 may as shown also comprise a handle housing 5a, and on the handle housing 5a a biopsy port 12 with a biopsy cap 12a for insertion of an external tool through the handle and the working channel to exit at the opening at the distal end of the endoscope. Further the handle 5 may be provided with buttons for activation of electrical switches 13 controlling image capture functions, such as taking of still images.

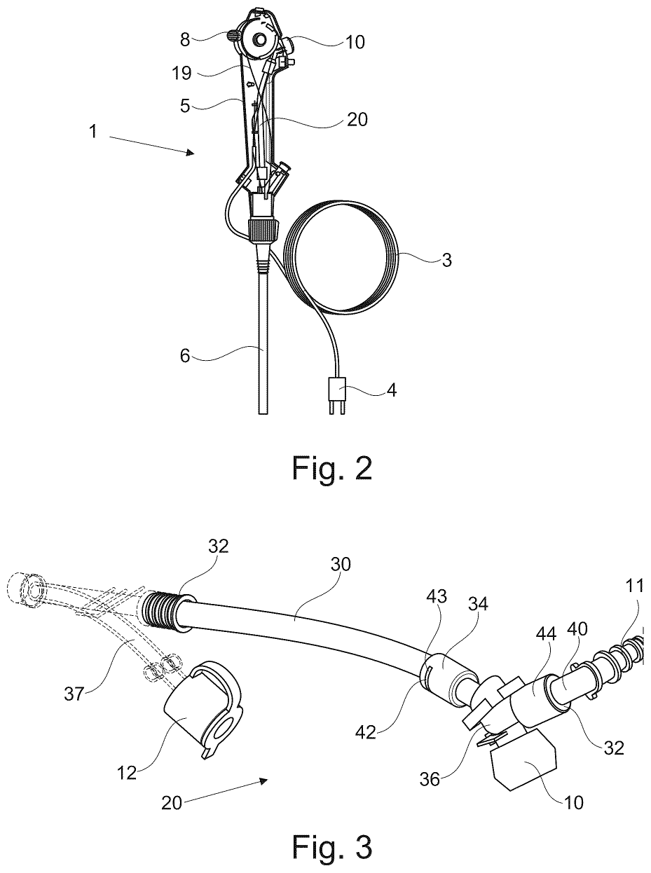

A view of the endoscope 1 with a portion of the handle housing removed to reveal the handle housing's interior is illustrated in FIG. 2. Steering wires 19 extend from the controller 8 to the bending section 7. A fluid pathway system 20 according to the present disclosure is arranged in the interior of the handle 5 and is discussed in more detail with reference to FIG. 3.

FIG. 3 illustrates the fluid pathway system 20 in isolation. The illustrated fluid pathway system 20 is coupled to a Y-connector 37 and comprises a valve 36, constituting an example of a first flow-through part, and a coupler of the Y-connector 37, constituting another example of a first flow-through part. The valve 36 comprises a tube coupler 34 at one end and a connector coupler 44 at the opposite end, both couplers configured to receive retainers 32. The couplers may be referred to as “receiver portions”. The valve 36 comprises the suction valve button 10 for activation of suction. A vacuum hose fitting or connector 11, constituting an example of a second flow-through part, has a connector part 40, which is inserted in the retainer 32, which again is inserted into the connector coupler 44 to form a fluidly sealed connection therewith. In turn, a suction tube 30, constituting another example of a second flow-through part, is inserted in the second retainer 32 to form a fluidly sealed connection with the tube coupler 34. The connector parts may be referred to as “tubular connection portions”.

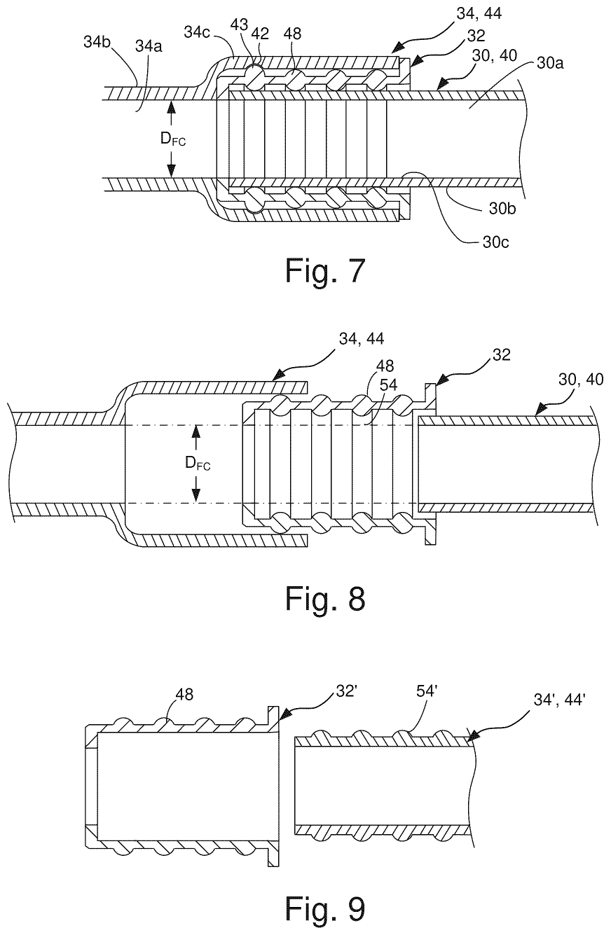

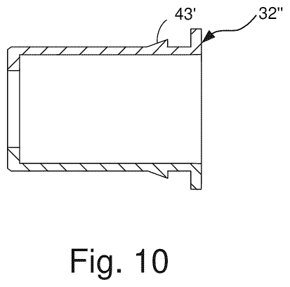

The first flow-through part may be provided with a retention recess and the retainer may be provided with a retention protrusion matching the retention recess to further secure anchoring of the retainer in the coupler. A retention recess 42 is shown, receiving a retention protrusion 43 of the retainer 32. The retention recess may be a through-hole or an indentation on an inner surface of the first flow-through part, for example. The through-hole may be an elongate hole, such as a slot. More than one retention recess and matching retention protrusion may be provided. From an assembly standpoint, the retainer may be inserted first to facilitate placement of the retention protrusion into the retention recess. Subsequently the second flow-through part is inserted into the retainer, preferably overlapping the retention protrusion to ensure it remains in place under pressure or tension. However, the retainer may be slid over the second flow-through part first and the combination may then be inserted into the first flow-through part, so long as the first flow-through part is not so rigid as to prevent insertion of a retainer with a retention protrusion. FIG. 7 illustrates an example of a retention recess 42 receiving a retention protrusion 43, where the retention recess 42 is an indentation, or slot (partially or completely circumferential), on the inner surface of the receiver portion, and where the retention protrusion 43 is a circular protrusion having a radial protrusion length greater than a gap between the inner surface of the receiver portion and the outer surface of the tubular connection portion. The retention protrusion may be biased to prevent dislodgment from the retention recess. For example, instead of an arcuate cross-section, the retention protrusion may comprise a slanted leading surface, to facilitate insertion, and a surface that is designed to require deformation of the retention protrusion before it can be dislodged. Such a retention protrusion may be referred to as a “removal deterrent retention protrusion.” FIG. 10 illustrates an example of a removal deterrent retention protrusion 43′ on a retainer 32″. Preferably, the removal deterrent retention protrusion 43′ is closer to the reception end of the receiver. This may facilitate removal by allowing easier deformation of the receiver to dislodge the removal deterrent retention protrusion 43′ from its corresponding retention recess. The retention recess may, similarly, comprise a trailing surface configured to prevent sliding removal of the deterrent retention protrusion 43′ without its deformation. Such a trailing surface may be a negative version of the trailing surface of the removal deterrent retention protrusion 43′.

The distal end of the suction tube 30 is inserted in a retainer 32, which is inserted in a coupler of a Y-connector 37, which constitutes another example of a second flow-through part.

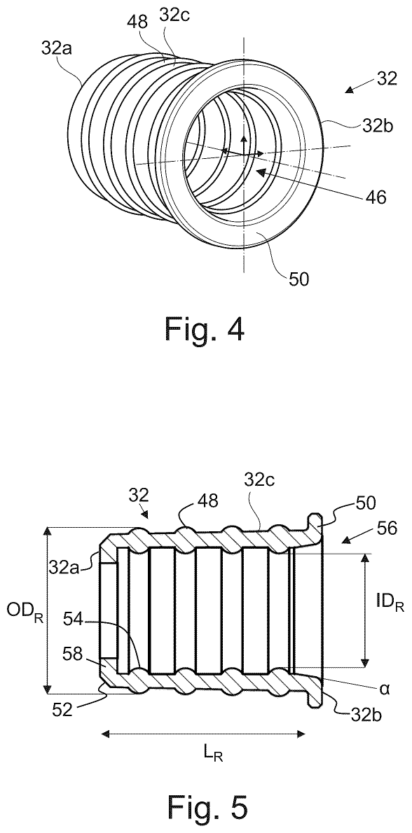

The retainer 32 will now be described in more detail with reference to FIGS. 4 and 5. As shown in FIG. 4, the retainer 32 comprises an insertion end 32a, a reception end 32b, and a wall 32c between the insertion end 32a and the reception end 32b, the wall 32c having a generally cylindrical shape defining a generally cylindrical inner space 46. The wall may comprise, optionally, a flange 50 at a reception end 32b and annular ribs 48 extending outwardly from an outer surface of the wall 32c.

Referring to FIG. 5, the insertion end 32a facilitates insertion in the coupler. By generally cylindrical inner space should be understood that the retainer 32 in a relaxed state thereof will have essentially the same inner diameter along the length of the retainer. However, protrusions may be provided on the inner surface of the wall 32c, such as annular ribs 54. Further, the entrance 56 to the inner space 46 may be slightly conical in shape as illustrated by angle α to facilitate insertion of the connector in the inner space. Additionally, the entry end 32a of retainer 32 may have a constriction 58 as shown, but is generally open with a through hole for the fluid flow.

The retainer 32 has a generally cylindrical outer shape and the generally cylindrical inner space 46. By generally cylindrical outer shape is meant that the retainer 32 in a relaxed state will have essentially the same outer diameter along the length of the retainer. However, protrusions may be provided on the outer surface, such as the annular ribs 48, and further the flange 50 may be provided at the reception end 32b as illustrated. The annular ribs 48 are semi-circular in cross-section, which is preferred at present, but it is considered that the protrusions may have any other shape, such as triangular or disk shaped. The retainer may further have a taper 52 at the insertion end 32a to facilitate insertion in the coupler. By generally cylindrical inner space should be understood that the retainer 32 in a relaxed state thereof will have essentially the same inner diameter along the length of the retainer. However, protrusions may be provided on the inner surface, such as annular ribs 54. Further, the entrance 56 to the inner space 46 may be slightly conical in shape as illustrated by angle α to facilitate insertion of the connector in the inner space. Additionally, the entry end 32a of retainer 32 may have a constriction 58 as shown, but is generally open with a through hole for the fluid flow.

As an example, the retainer may have a total length of 10-15 mm, such as 12.85 mm. The retainer may have an outer diameter, including any potential protrusions, of 9-11 mm, such as 9.55 mm. The retainer may have an inner diameter, taking any potential protrusions into account, of 5-8 mm, such as 6.65 mm. A wall thickness of the retainer between any potential protrusions may be 0.75 mm to 1 mm, such as 0.89 mm. A wall thickness at potential protrusions may be 1.5 to 2.5 times the wall thickness between protrusions, such as approximately 1.8 times the wall thickness between protrusions. A through hole at the entry end 32a of the retainer 32 may have a diameter of 5-6 mm, such as 5.5 mm. The outer diameter of the flange (if any), may be 10-12 mm, such as 11.23 mm.

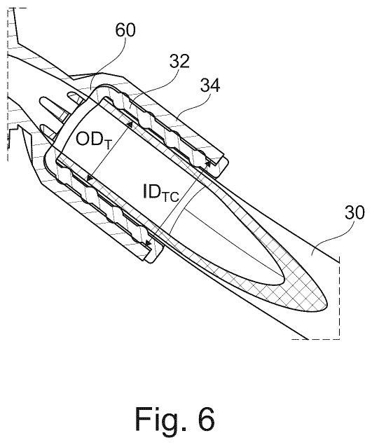

FIG. 6 is a perspective view of a sectioned assembled fluid pathway system, namely a longitudinal section of the suction tube 30 fitted in the retainer 32, which in turn is fitted in the coupler 34 of the valve 36 (see FIG. 3). The retainer 32 is configured to fit the interior of the coupler 34, and likewise the retainer 32 is configured to fit the exterior of the suction tube 30. In this embodiment the length of the retainer 32 corresponds to the length of the interior of the coupler 34, so the end of the retainer 32 abuts a step 60 of the coupler 34. The constriction 58 may act as a seal between the end of the suction tube 30 and the step 60 of the coupler 34. The coupler 34 is generally shell shaped, but it is conceivable to provide barbs or cut-outs in the inner surface of the coupler 34 for increased grip between the coupler 34 and the retainer 32, such as the cut-out 42 discussed above or with reference to FIG. 7. As can be seen, the fluid flow path through the assembled fluid pathway system, referred to as D_{FC} (e.g. fluid channel diameter) is virtually unrestricted and smooth with the retainer constriction having an inner opening diameter configured to correspond to the inner diameter of other parts making up the fluid pathway system, such as the inner diameter of the suction tube, so the pressure loss is kept at a minimum.

FIGS. 7 to 9 illustrate some of the features described above. FIG. 7 is a side view of an example of the assembled fluid pathway system, comprising: a first flow-through part 34, 44 having a fluid conduit 34a therethrough in tubular portion 34b, and comprising a receiver portion 34c, a second flow-through part 30, 40 having a fluid conduit 30a therethrough in tubular portion 30b and comprising a tubular connector portion 30c, a retainer 32 including an insertion end and a reception end, the insertion end of the retainer being inserted in the receiver portion of the first flow-through part, the tubular connector portion of the second flow-through part being inserted in the reception end of the retainer, and the retainer being sized and configured to fluidly seal the second flow-through part to the first flow-through part without adhesives.

A wall extends between an insertion end and a reception end of the retainer. Such fluid sealing may be performed by the inclusion of annular sealing protrusions on the inner surface and/or the outer surface of the wall. The fluid pathway system may be fluidly sealed without reliance on adhesives to form the seal. Adhesives may be used to form the first or second flow-through parts, however, thus the fluid pathway system can be said to be essentially adhesive-free in the sense that the adhesives do not form the seal. For example, a flow-through part may be extruded or injection molded in one part or two parts that are bonded together. A retention recess 42 is shown in the inner surface of the receiver portion, receiving a retention protrusion 43 of the retainer 32. In this example the retaining protrusion 43 is also an annular sealing protrusion.

FIG. 8 is an exploded view of the fluid pathway system as shown in FIG. 6. FIG. 9 is an exploded view of portions of another example of the fluid pathway system illustrating that annular sealing protrusions 54′ can also be provided on the outer surface of the tubular connector portion 34′, 44′. Providing annular sealing protrusions on the outer surface of the tubular connector portion instead of the inner surface of the retainer 32′ might reduce manufacturing costs by simplifying molds for injection molding since only cylindrical mandrels are necessary to form the cavities. Of course, annular sealing protrusions can be provided on the outer surface of the tubular connector portion in addition to the annular sealing protrusions provided on the inner surface of the retainer.

Single-use endoscopes optimize workflow and reduce cost while saving patient's lives and improving patient care. They optimize workflow and reduce cost because they are always ready when needed without the traditional large-scale capital and repair budgets required for reusable endoscopes. For example, a sterilization and storage facility is avoided, there is no need to maintain evidence of sterilization, and there is no need to transport endoscopes from sterilization and storage facilities to the buildings where they are needed, sometimes in the middle of the night or weekends. They save patient's lives and improve patient care because they are readily available and do not pose a cross-contamination risk. This also reduces hospital re-admissions. While single-use endoscopes are disposed after a single patient use (one or more procedures may be performed while the patient remains in the treatment room), the environmental impact of re-useable endoscopes, due to cleaning materials, CO₂ emissions during the cleaning process, and use of disposable personal protective equipment by personnel involved in transportation and sterilization of the re-useable endoscopes, is similar to that of single-use endoscopes. Studies are emerging showing that the environmental impact of single-use endoscopes may, in fact, be less than that of re-usable endoscopes. To further reduce environmental impact, the endoscopes according to the present disclosure are primarily made of polymer materials.

The following items are further variations and examples of the embodiments described with reference to the figures.

1. An endoscope comprising: a handle or interface, an insertion cord extending distally from the handle or interface, and a fluid pathway system comprising: a first flow-through part having a fluid conduit formed therein and comprising a receiver portion, a second flow-through part having a fluid conduit formed therein and comprising a tubular connector portion, a retainer having an insertion end and a reception end, wherein the insertion end of the retainer is inserted in the receiver portion of the first flow-through part, wherein the tubular connector portion of the second flow-through part is inserted in the reception end of the retainer, and wherein the retainer is sized and configured to fluidly sealingly secure the second flow-through part to the first flow-through part.

2. The endoscope according to item 1, wherein the receiver part has a generally cylindrical shape and a receiver inner diameter, wherein the tubular connector portion has a connector outer diameter, wherein the retainer has a generally cylindrical outer surface and an inner space having a generally cylindrical inner surface, wherein the retainer has an inner retainer diameter of the inner space and an outer retainer diameter of the outer surface, the retainer inner diameter fitting the connector portion outer diameter and the retainer outer diameter fitting the receiver portion inner diameter to form a fluid seal between the second flow-through part, the retainer, and the first flow-through part.

3. The endoscope according to any one of the items above, wherein the retainer comprises annular sealing protrusions on the inner surface and/or the outer surface.

4. The endoscope according to any one of the items above, wherein the first flow-through part is a Y-connector, and the second flow-through part is a suction tube.

5. The endoscope according to any one of the items above, wherein the first flow-through part is a suction valve, and the second flow-through part is a suction tube and/or a vacuum hose fitting.

6. The endoscope according to any one of the items above, wherein the fluid pathway system is essentially adhesive-free.

7. The endoscope according to any one of the items above, wherein the retainer is made of a material having a hardness in the interval of 10-100 Shore A, preferably 50-70 Shore A.

8. The endoscope according to any one of the items above, wherein the retainer comprises a flange at the receiver end thereof.

9. The endoscope according to any one of the items above, wherein the retainer comprises a lead-in zone to the inner space at the receiver end.

10. The endoscope according to any one of the items above, wherein the retainer comprises a constriction at the insertion end thereof.

11. The endoscope according to item 10, wherein the inner interface between first flow-through part, second flow-through part and retainer constriction is configured to provide a consistent inner diameter.

12. The endoscope according to any one of the items above, wherein the retainer has a ratio of length to outer diameter in the range of 6:5 to 7:5.

13. The endoscope according to any one of the items above, wherein the retainer is molded in one piece from an elastic material, such as medical grade silicone.

14. The endoscope according to any one of the items above, wherein the retainer comprises a removal deterrent retention protrusion.

15. The endoscope according to item 15, wherein the removal deterrent retention protrusion comprises a slanted leading surface configured to facilitate insertion and a removal deterring trailing edge.

16. The endoscope according to item 16, wherein the removal deterrent retention protrusion is positioned closer to the receiving end than to the insertion end of the retainer.

17. The endoscope according to item 1, wherein the fluid pathway system is assembled without adhesives, wherein the retainer is comprised substantially of medical grade silicone, wherein the retainer comprises a removal deterrent retention protrusion, and wherein the retainer is devoid of annular sealing protrusions.

18. The endoscope according to item 1, wherein the fluid pathway system is assembled without adhesives, wherein the retainer is comprised substantially of medical grade silicone, and wherein the retainer is devoid of annular sealing protrusions.

19. A visualization system comprising: an endoscope according to item 1; and a monitor connectable to the endoscope.

### LIST OF REFERENCE SIGNS

1 endoscope
1a system
2 display unit
3 umbilical cord
4 electrical connector
5 handle
6 insertion cord
7 bending section
8 controller
9 tip part
10 suction valve button
11 vacuum hose connector
12 biopsy connector
12a biopsy cap
13 electrical switch
14 suction tube
16 suction valve
16a tube connector
17 gap
18 Y-connector
18a tube connector
19 steering wire
20 fluid pathway system
30 suction tube
32 retainer
32a insertion end
32b reception end
34 tube coupler
36 valve
37 Y-connector
40 connector part
42 cut-out
43 retention protrusion
44 coupler
46 inner space
48 annular ribs
50 flange
52 taper
54 annular ribs
56 entrance
58 constriction
60 step
IDR Inner diameter, retainer
ODR Outer diameter, retainer
LR Length, retainer
IDTC Inner diameter, tube coupler
ODT Outer diameter, tube
α angle

1. An endoscope comprising:
a handle or interface;
an insertion cord extending distally from the handle or interface; and
a fluid pathway system comprising:
a first flow-through part having a fluid conduit therethrough and comprising a receiver portion;
a second flow-through part having a fluid conduit therethrough and comprising a tubular connector portion;
a retainer having an insertion end and a reception end, the insertion end of the retainer being inserted in the receiver portion of the first flow-through part, and the tubular connector portion of the second flow-through part being inserted in the retainer, the retainer being sized and configured to fluidly sealingly secure the second flow-through part to the first flow-through part without adhesives.

2. The endoscope of claim 1, wherein the retainer comprises a wall extending between the insertion end and the reception end and annular sealing protrusions extending radially from the inner surface and/or the outer surface of the wall.

3. The endoscope of claim 1, wherein the retainer comprises a wall extending between the insertion end and the reception end, annular sealing protrusions extending radially from the inner surface and/or the outer surface of the wall, and a retention protrusion, and wherein the receiver portion comprises a retention recess receiving the retention protrusion to further secure the retainer to the first flow-through part.

4. The endoscope of claim 3, wherein the retention recess is a through-hole.

5. The endoscope of claim 1, wherein the first flow-through part is a suction valve, wherein the second flow-through part comprises a suction tube fluidly sealingly connected to the receiver portion of the suction valve, wherein the suction valve further comprises a second receiver portion, and wherein the endoscope further comprises a vacuum hose connector fluidly sealingly connected to the second receiver portion of the suction valve.

6. The endoscope of claim 1, wherein the receiver portion has a generally cylindrical shape and a receiver inner diameter, wherein the tubular connector portion has a connector outer diameter, wherein the retainer has a generally cylindrical outer surface and an inner space having a generally cylindrical inner surface, wherein the retainer has an inner retainer diameter of the inner space and an outer retainer diameter of the outer surface, the retainer inner diameter fitting the connector portion outer diameter and the retainer outer diameter fitting the receiver portion inner diameter to form a fluid seal between the second flow-through part, the retainer, and the first flow-through part.

7. The endoscope of claim 6, wherein the retainer comprises annular sealing protrusions on the inner surface and/or the outer surface.

8. The endoscope of claim 1, wherein the first flow-through part is a Y-connector, and the second flow-through part is a suction tube.

9. The endoscope of claim 1, wherein the first flow-through part is a suction valve, and the second flow-through part is a suction tube and/or a vacuum hose fitting.

10. The endoscope of claim 1, wherein the fluid pathway system is essentially adhesive-free.

11. The endoscope of claim 1, wherein the retainer is made of a material having a hardness in the interval of 10-100 Shore A.

12. The endoscope of claim 11, wherein the hardness is in the interval of 50-70 Shore A.

13. The endoscope of claim 1, wherein the retainer comprises a flange at the receiver end thereof.

14. The endoscope of claim 1, wherein the retainer comprises a lead-in zone to the inner space at the receiver end.

15. The endoscope of claim 1, wherein the retainer comprises a constriction at the insertion end thereof.

16. The endoscope of claim 15, wherein an inner interface between first flow-through part, second flow-through part and the retainer constriction is configured to provide a consistent inner diameter.

17. The endoscope of claim 1, wherein the retainer has a ratio of length to outer diameter in the range of 6:5 to 7:5.

18. The endoscope of claim 1, wherein the retainer is molded in one piece from an elastic material.

19. The endoscope of claim 1, wherein the retainer is molded in one piece a medical grade silicone.

20. A visualization system comprising:
the endoscope of claim 1; and
a monitor connectable to the endoscope.
